(19) United States
(12) Patent Application Publication
(10) Pub. No.: US 2007/0270849 A1
(43) Pub. Date: Nov. 22, 2007

(54) Fixation Plate With Multifunctional Holes

(76) Inventors: Jorge L. Orbay, Miami, FL (US); Cesare Cavallazzi, Miramar, FL (US); Edward Mebarak, Miami, FL (US); Christopher Kelly Bremer, Warsaw, IN (US)

Correspondence Address: GORDON & JACOBSON, P.C., 60 LONG RIDGE ROAD, SUITE 407, STAMFORD, CT, 06902, US

(21) Appl. No.: 11/379,646

(22) Filed: Apr. 21, 2006

(51) Int. Cl. A61F 2/30

(52) U.S. Cl. 606/69

(57) ABSTRACT

A bone plate for internal fixation of a bone fracture includes top and bottom surfaces and an edge. At least one multifunctional hole is provided along the edge and communicates between the top and bottom surfaces and the edge. The hole generally includes a cylindrical portion for closely receiving and guiding a K-wire in a fixed angle. The hole also includes a lateral channel formed into the bottom surface and the edge of the plate such that a curved suture needle and attached suture material may be passed through the hole even when the plate is fixed to the bone. The multifunctional holes may be arranged along the edges of the plate to maintain or modify the stiffness of the plate.

### BACKGROUND OF THE INVENTION

1. Field of the Invention

This invention relates broadly to surgical devices. More particularly, this invention relates to a fracture fixation plate that may be used with K-wires and/or suture material.

2. State of the Art

Fracture fixation plates are common in the industry. Such plates are use to bridge across fractures, stabilizing fragments for healing. Fixation plates may be in the form of fragment plates which are generally used along the diaphysis of long bones or anatomically specific metaphyseal plates which are contoured for the articulating end of long bones. The plates are generally provided with a plurality of screw holes at which the plates can be secured to the bone with screws or other fasteners. The holes may be locking holes, non-locking holes, combination holes (which can be locking or non-locking depending upon the fastener used therethrough), and a combination of any of the above.

It is common for fixation plates to include K-wire holes. Stiff K-wires are often used to temporarily secure a plate to the bone prior to and during fixation by the fasteners. The K-wires are then removed.

In addition, it is known to provide fixed angle K-wire holes that have a cylindrical bore and which are sized to closely fit with K-wires so as to guide K-wires in a predefined angular orientation. Such orientation is closely related to the angle of one or more of the fasteners such that that path of the inserted K-wires anticipates the path of the fasteners. Then, when viewing the plate and inserted K-wires under fluoroscopy, the surgeon is provided with visual indication of whether the fasteners, once inserted, will be properly aligned with the anatomy. See, e.g., US Pub. No. 20050065524 A1 to Orbay.

Furthermore, depending upon the surgical procedure, the surgeon may need to incise the insertions of certain muscles to facilitate reduction of the fractured bone and to provide access for attachment of the plate to the bone. For example, in a procedure to fixate a proximal humeral fracture, the deltoid and pectoralis muscle insertions are commonly incised and after fixing the plate to the bone, the deltoid and pectoralis tendons may be fixed to the plate to reconstruct the musculature. As such, it is known to provide a plate with suture holes for receiving therethrough a suture needle and suture material, e.g., cord or cable. Suture holes are designed differently than K-wires holes. Rather than have structure for fixed angle guidance, the suture holes are heavily chamfered for prevent damage to the suture material.

Thus, fixation plates are often replete with holes with dedicated functionality. Such holes crowd the plate surface, often making it difficult for a surgeon to identify which holes are to be used for which purpose. In addition, there may not be sufficient space on the plate to place the holes at the optimal locations for the desired purposes. In Furthermore, the shear number of holes dedicated to each function may result in a plate that is weaker than desired for the particular application.

U.S. Pub. No. 20050261688 to Grady, Jr. et al. discloses a plate having holes that are indicated to be for either guide wires or suture, thereby reducing the total number of holes. However, there is no disclosure that the holes are intended to guide stiff wires at a fixed angle. In addition, the design of the holes does not permit a suture needle to be inserted between the bone and the bone contacting surface of the plate after the plate is positioned against or attached to the bone. Suture holes which permit suture needle passage after the plate is fixed to the bone generally include a bottom clearance for the needle, as disclosed, e.g., by the Humeral SuturePlate™ from Arthrex Inc. of Naples, Fla.

### SUMMARY OF THE INVENTION

It is therefore an object of the invention to provide a fracture fixation plate having a multifunctional hole that accommodates a K-wire at a fixed angle and which allows a suture needle to be inserted through the hole even after the plate has been fixed to the bone.

It is another object of the invention to provide a multifunctional hole that reduces the total number of holes required in a plate, thereby maintaining desired plate strength and allowing optimal placement of the holes within the plate.

It is also an object of the invention to provide multifunctional holes to the plate in an arrangement that maintain the longitudinal and torsional stiffness of the plate.

It is a further object of the invention to provide multifunctional holes to the plate in an arrangement that increases flexibility of the plate at specific locations.

It is yet another object of the invention to provide a multifunctional hole configuration that minimizes stress concentration to suture extending therethrough.

It is an object of the invention to provide such multifunctional holes to both metaphyseal plates and diaphyseal plates.

In accord with these objects, which will be discussed in detail below, a bone plate for internal fixation of a bone fracture includes top and bottom surfaces and an edge. At least one multifunctional hole is provided along the edge and communicates between the top and bottom surfaces. The hole generally includes a cylindrical portion for closely receiving and guiding a stiff K-wire in a fixed angle. The hole also includes a lateral channel or recess formed into the bottom surface and an edge of the plate such that a curved suture needle and attached suture material may be passed through the hole and out of the side of the plate even when the plate is fixed to the bone. A plurality of multifunctional holes may be arranged along the edges of the plate, either closely spaced, e.g. in a metaphyseal plate, or spaced apart along sides of a diaphyseal plate. If arranged in a staggered manner along opposed sides, the plate maintains its longitudinal and torsional stiffness, whereas if the multifunctional holes are in an opposing arrangement, the plate develops areas of decreased stiffness at which the plate can bend to conform to the anatomy when coupled to the bone.

Additional objects and advantages of the invention will become apparent to those skilled in the art upon reference to the detailed description taken in conjunction with the provided figures.

### BRIEF DESCRIPTION OF THE DRAWINGS

FIG. 1 is a top view of a first embodiment of a bone plate including a multifunctional hole according the invention;

FIG. 2 is a side elevation view of the first embodiment of the bone plate;

FIG. 3 is a bottom view of the first embodiment of the bone plate;

FIG. 4 is an enlarged top view of a multifunctional hole according to the invention in the bone plate;

FIG. 5 is an enlarged side elevation of the multifunctional hole;

FIG. 6 is an enlarged bottom view of the multifunctional hole;

FIG. 7 is a section view across line 7-7 in FIG. 1;

FIG. 8 is a view similar to FIG. 7, shown with the bone plate positioned on bone and a K-wire held within a fixed-angle with the multifunctional hole;

FIG. 9 is a view similar to FIG. 7, shown with the bone plate positioned on bone and a suture needle extending through the multifunctional hole;

FIG. 10 is a top view of a second embodiment of a bone plate including a multifunctional hole according the invention;

FIG. 11 is a bottom view of the second embodiment of the bone plate;

FIG. 12 is a section view across line 12-12 in FIG. 10;

FIG. 13 is a perspective view of a third embodiment of a bone plate including a multifunctional hole according the invention;

FIG. 14 is a top view of the third embodiment of a bone plate;

FIG. 15 is a longitudinal section view of the third embodiment of the bone plate; and

FIG. 16 is a bottom view of the third embodiment of the bone plate.

### DETAILED DESCRIPTION

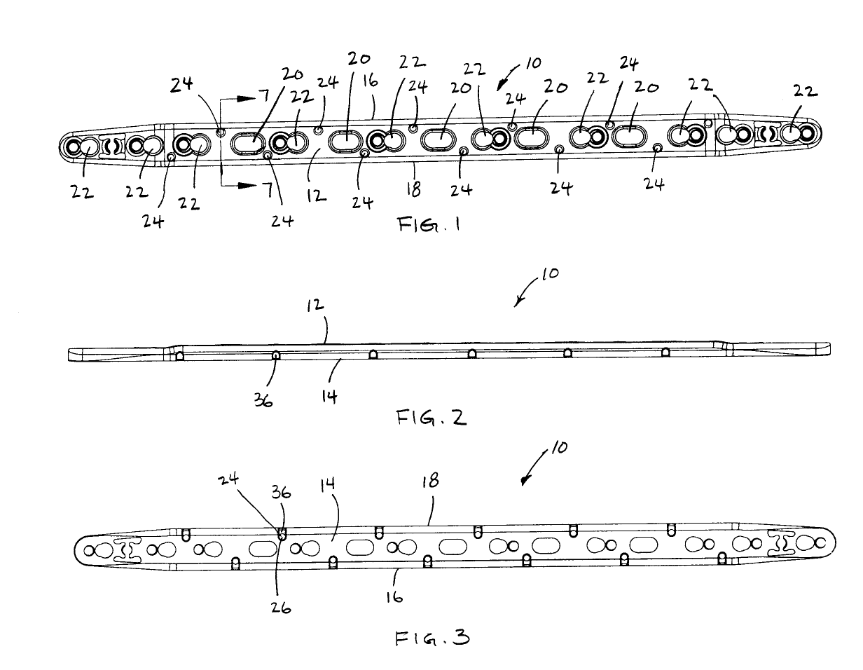

Turning now to FIGS. 1 through 3, a first embodiment of bone plate 10 for internal fixation of a bone fracture is shown. The plate is an elongate diaphyseal plate for fractures along a shaft portion of a long bone. The plate includes top and bottom surfaces 12, 14 and edges 16, 18 thereabout. Between the top and bottom (bone contacting) surfaces and along the length of the plate a plurality of screw holes 20, 22 are provided. Such screw holes may be variable angle, fixed angle, or combination variable and fixed angle screw holes. Commonly known fixators, such as, e.g., fixed angle screws, variable angle screws, locking screws, and locking pegs, may be provided for use through the holes 20, 22.

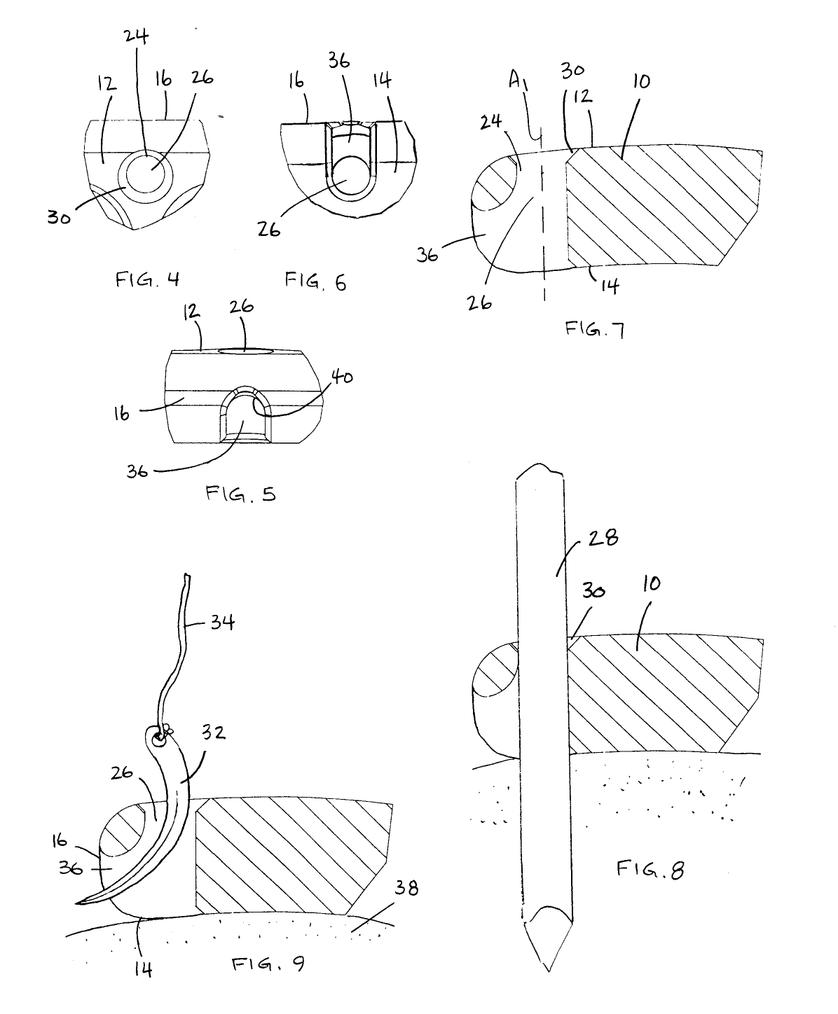

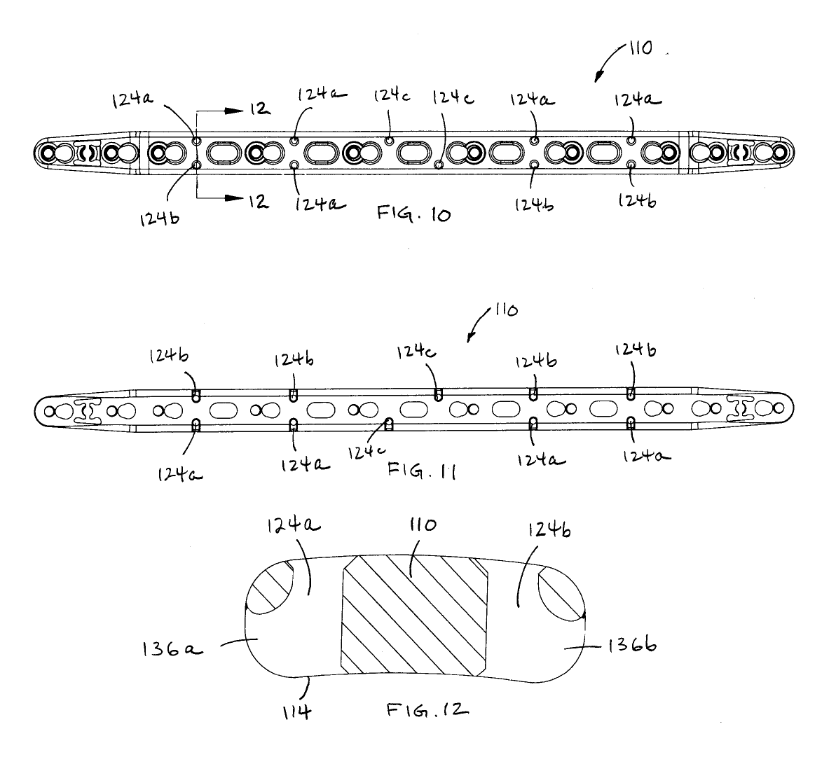

Referring to FIGS. 2 through 9, a plurality of multifunctional holes 24 for both K-wire and suture needle/suture material is also provided along the length of the plate adjacent the edges 16, 18 and communicate between the top and bottom surfaces 12, 14. Each multifunctional hole 24 generally includes a cylindrical portion 26 sized for closely receiving and guiding substantially along fixed axis A₁ (e.g., ±3°) an appropriately sized stiff K-wire 28 (FIG. 8). The axis A₁ is generally approximately normal to the top and/or bottom surfaces 12, 14 of the plate. As an alternative, portion 26 can be non-cylindrical, but otherwise shaped for substantially fixed-angle guidance of the K-wire along axis A₁. For example, the portion 26 may be provided with a regular polygonal cross-sectional (hexagonal, octagonal, etc.) which also guides the K-wire in a substantially fixed angle, as described. The cylindrical portion 26 has a circular upper opening 30 which is slightly flared at its entry to facilitate introduction of the K-wire 28 (FIG. 8) or suture needle 32 (FIG. 9, discussed below) and prevent stress concentration on suture material 34. The multifunctional hole 24 also includes a lateral channel (or lower recessed portion) 36 (i.e., directed toward the adjacent edge 16 of the plate) formed into the bottom surface 14 and the adjacent edge 16 of the plate such that a curved suture needle 32 and attached suture material 34 may be passed through the hole and laterally out of the side of the plate even when the plate is fixed to bone 38. The lateral channel 36 preferably assumes approximately half the height of the plate. The surface of the lateral channel 36 is defined by the inner surface of a toroidal section; i.e., suture guide surface 40 is concave across its width, as shown at the exit of the channel 36 in FIG. 5. In addition, all surfaces along the suture channel are broken or radiused. Thus, when suture material 34 is passed through the plate, stress concentrations on the suture material is limited and the likelihood of suture material breakage is minimized.

Referring back to FIGS. 1 and 3, a plurality of the multifunctional holes 24 may be arranged adjacent the edges 16, 18 of the plate 10 in staggered arrangement. The alternating arrangement substantially maintains the longitudinal and torsional stiffness of the plate.

Turning now to FIGS. 10 through 12, according to second embodiment of the invention, it is also recognized that the multifunctional holes 124a, 124b may be spaced apart along the fixation plate 110 in an arrangement of opposed pairs. At each opposed pair of multifunctional holes 124a, 124b, the plate develops areas of decreased longitudinal stiffness as a result of the opposed lateral recesses 136a, 136b at the bottom surface 114 of the plate. By way of example, FIG. 12 is a view across an area of decreased longitudinal stiffness. The decreased stiffness at such areas allows the plate to bend to conform to the shape of the bone when the plate is attached to the bone with fixators. Such plate may additionally include staggered holes 124c as well.

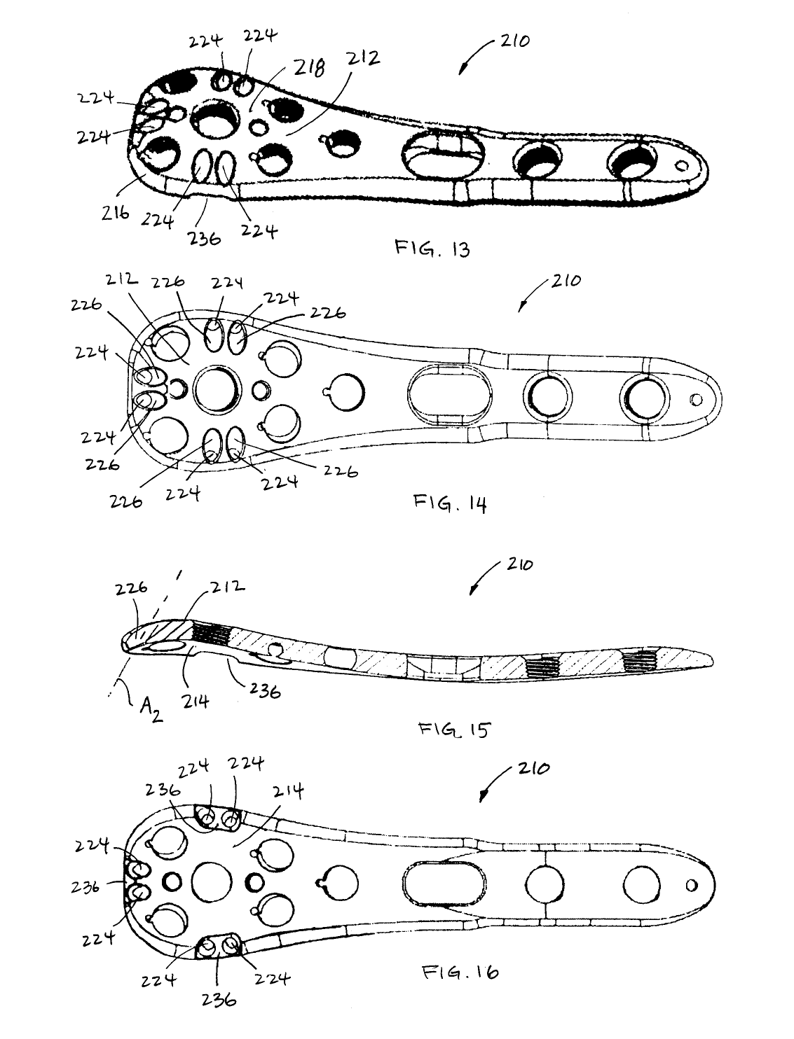

Referring now to FIGS. 13 through 16, a third embodiment of the invention is shown. The specific plate 210 shown, by way of example, is a metaphyseal plate for fixation of humeral fractures. The plate includes top and bottom surfaces 212, 214. In plate 210, three closely spaced pairs of multifunctional holes 224 are shown located around the peripheral edge 216 of the head portion 218 of the plate 210. The cylindrical K-wire guiding portion 226 of a multifunctional hole is oriented at an oblique angle along axis A₂ relative to the top and bottom surfaces 212, 214 of the plate. At the bottom surface 214 of the plate a wide lateral channel or recess 236 is provided and encompasses the exits of both K-wire guiding portions 226 of the holes.

There have been described and illustrated herein several embodiments of a fixation plate. While particular embodiments of the invention have been described, it is not intended that the invention be limited thereto, as it is intended that the invention be as broad in scope as the art will allow and that the specification be read likewise. Thus, while particular embodiments of diaphyseal and metaphyseal plates with multifunctional holes according to the invention have been disclosed, it will be appreciated that the multifunctional holes can be located in plates of other shapes and sizes and which are otherwise adapted for use on various bones. In addition, the locations for the multifunctional holes within the plates can be modified as well, while still being within the scope of the invention. Specifically, a combination of staggered and opposed pairs of multifunctional holes, and more or fewer multifunctional holes (including single holes), spaced relatively closer or farther apart than shown in the exemplar embodiments are within the scope of the invention. Furthermore, while it is preferred that the guide portion guide a K-wire at a fixed angle, it is appreciated that the guide portion may be sized or otherwise configured to guide the K-wire at various angles between the top and bottom surfaces of the plate. It will therefore be appreciated by those skilled in the art that yet other modifications could be made to the provided invention without deviating from its scope as claimed.

What is claimed is:

1. A bone plate for fixation of a bone fracture, comprising:
a bone plate for internal fixation of a bone fracture, said plate including top and bottom surfaces and at least one edge thereabout,
said plate defining a plurality of screw holes extending between said top and bottom surfaces for receiving fixators therethrough to couple said plate to the bone, and
said plate defining at least one multifunctional hole, said multifunctional hole including a wire guide portion which is structured to guide a stiff wire at a substantially fixed angle relative to said plate and a lateral recess extending from said guide portion to an adjacent edge, said lateral recess open to said bottom surface of said plate.

2. A bone plate according to claim 1, wherein:
said guide portion is cylindrical.

3. A bone plate according to claim 1, wherein:
said guide portion defines an axis that is normal to at least one of the top and bottom surfaces of said plate.

4. A bone plate according to claim 1, wherein:
said guide portion defines an axis that is oblique relative to at least one of the top and bottom surfaces of said plate.

5. A bone plate according to claim 1, wherein:
said guide portion has a flared opening at said top surface.

6. A bone plate according to claim 1, wherein:
said plate has a height, and at said adjacent edge said lateral recess assumes approximately half said height of said plate.

7. A bone plate according to claim 1, wherein:
said lateral recess includes a curved suture guide surface.

8. A bone plate according to claim 1, wherein:
said exit of said lateral recess at said adjacent edge is concavely curved.

9. A bone plate according to claim 1, wherein:
said plate includes a length, and a plurality of said multifunctional holes are spaced apart along said length.

10. A bone plate according to claim 1, wherein:
at least a plurality of said multifunctional holes are in a staggered arrangement along said length.

11. A bone plate according to claim 1, wherein:
said plate includes opposite edges, and
at least a plurality of said multifunctional holes along said length are arranged in pairs, wherein one of each of said pair is located along an opposite edge of said plate directly across from its pair.

12. A bone plate according to claim 1, wherein:
said plate is a diaphyseal plate.

13. A bone plate according to claim 1, wherein:
said plate is a metaphyseal plate.

14. A bone plate for fixation of a bone fracture, said plate for use with a stiff wire and a suture needle, said plate comprising:
a bone plate for internal fixation of a bone fracture, said plate including top and bottom surfaces and a peripheral edge thereabout,
said plate defining a plurality of screw holes extending between said top and bottom surfaces for receiving fixators therethrough to couple said plate to the bone, and
said plate defining,
a first wire guide structured to guide the stiff wire at a substantially fixed angle relative to said plate,
a second wire guide structured to guide the stiff wire at a substantially fixed angle relative to said plate,
each of said first and second wire guides having an entry and an exit, and
a recess extending from said exits of said first and second guides to said peripheral edge,
wherein the suture needle may be passed through either of said first and second wire guides and exit laterally of said plate through said recess after said bottom surface of said plate has been coupled substantially flush against the bone.

15. A bone plate according to claim 14, wherein:
at least one of said first and second wire guides defines a fixed angle axis for the stiff wire that is oblique relative to one of said top and bottom surfaces of said plate.

16. A bone plate according to claim 14, wherein:
said plate is a metaphyseal plate including a head portion and said first and second wire guides and recess are provided together in said head portion.

17. A bone plate according to claim 16, wherein:
a plurality of combinations of said first and second wire guides and recess are spaced apart about a periphery of said head portion.

18. A bone plate for fixation of a bone fracture, comprising:
a bone plate for internal fracture fixation, said plate including at least one multifunctional hole including both guide means for guiding a stiff wire at a substantially fixed angle and needle exit means for exiting a curved suture needle out of said multifunctional hole and out of a periphery of said plate after said plate has been coupled substantially flush against the bone.

19. A bone plate according to claim 18, wherein:
said bone plate includes top and bottom surfaces, and said guide means is for guiding the stiff wire along an axis that is normal to at least one of said top and bottom surfaces.

20. A bone plate according to claim 18, wherein:
said bone plate includes top and bottom surfaces, and said guide means is for guiding the stiff wire along an axis that is oblique to at least one of said top and bottom surfaces.

21. A bone plate according to claim 18, wherein:
said plate includes a length, and a plurality of said multifunctional holes are spaced apart along said length.

22. A bone plate according to claim 21, wherein:
at least a plurality of said multifunctional holes are in a staggered arrangement along said length.

23. A bone plate according to claim 21, wherein:
said plate includes opposite edges, and
at least a plurality of said multifunctional holes along said length are arranged in pairs, wherein one of each of said pair is located along an opposite edge of said plate directly across from its pair.

24. A bone plate for fixation of a bone fracture, comprising:
a bone plate for internal fixation of a bone fracture, said plate including top and bottom surfaces and at least one edge thereabout,
said plate defining a plurality of screw holes extending between said top and bottom surfaces for receiving fixators therethrough to couple said plate to the bone, and
said plate defining at least one multifunctional hole, said multifunctional hole including a wire guide portion which is structured to receive a stiff wire through said top and bottom surfaces of said plate and a lateral recess extending from said guide portion to an adjacent edge, said lateral recess open to said bottom surface of said plate.
